(19) United States
(12) Patent Application Publication
(10) Pub. No.: US 2016/0262844 A1
(43) Pub. Date: Sep. 15, 2016

(54) METHOD AND SYSTEM FOR DETECTING AND MARKING METALLIC MATERIALS ON AND INSIDE THE HUMAN BODY

(71) Applicant: NEXUS CONTROL SYSTEMS, LLC, PORT WASHINGTON, NY (US)

(72) Inventors: TODD J. COHEN, PORT WASHINGTON, NY (US); JOHN R. LUBISICH, WEST LINN, OR (US)

(73) Assignee: NEXUS CONTROL SYSTEMS LLC, PORT WASHINGTON, NY (US)

(21) Appl. No.: 15/065,595

(22) Filed: Mar. 9, 2016

Related U.S. Application Data
(60) Provisional application No. 62/130,070, filed on Mar. 9, 2015.

(51) Int. Cl. A61B 90/00

(52) U.S. Cl. CPC A61B 90/39; A61B 2090/3925; A61B 2090/3937; A61B 2090/3954

(57) ABSTRACT

A handheld medical system detects at least one object within a patient's body based on an audible and/or visual feedback system, prior to surgical exploration, removal, revision, or replacement.

### CROSS-REFERENCE TO RELATED APPLICATION

This application is based upon and claims the benefit of the priority of the filing date of U.S. Provisional Patent Application Ser. No. 62/130,070, filed Mar. 9, 2015, which application is incorporated herein by reference in its entirety

### FIELD OF THE INVENTION

The invention described herein relates to a method and system for detecting or identifying metallic materials that are on or within the human body. More particularly, this invention provides a system and method for sterile scanning and for marking on the skin the location of metallic and nonmetallic materials that reside on or under the skin in preparation for surgical removal or explantation. In addition, the invention provides a method of scanning a patient prior to wound closure to avoid the complication of a retained foreign body. Further, the sterile handheld device can be used post-surgery to identify and locate a retained foreign body.

### BACKGROUND OF THE INVENTION

At present there is no simple and inexpensive sterile medical device and/or system that can be used to scan the human body and detect or identify difficult to find materials, metallic or nonmetallic, within the human body that may require removal, revision, replacement, and/or extraction. With some larger devices, such as implantable defibrillators, it is easy to find a subcutaneous device because of its size, mass, and location of placement. Up until recently, smaller devices, such as a pacemaker, could usually be identified. However, newer systems and technology are often so small that location during surgery—especially after sterile draping of the patient—may be difficult. Operators have experienced difficulty finding a paper clip size subcutaneous metallic loop recorder and can inadvertently hit an artery, which could contribute to bleeding. This can occur even with the presence of X-ray equipment such as a fluoroscope and other surgical exploratory tools. Thus, there is a need to precisely locate and mark the skin, prior to the surgical exploration, and particularly in a manner which does not break sterility during the surgical procedure.

Currently, hidden medical implants and other metal objects can be identified via X-ray methodology prior to surgical removal, revision, extraction, explantation, or the like. This methodology exposes the patient to potentially harmful X-rays. In addition, the operator has to wear heavy X-ray shielding equipment (for example, lead aprons) during the surgery. In addition, this methodology is often not precise, and a metal or radio-opaque tool also needs to be utilized to help locate the metallic object beneath the X-ray beam.

Alternatively, skin scars and marks from prior wounds can help to show the initial location in which an object may have traversed the skin prior to its final resting site within the human body. Blindly opening such a site and exploring for the metal device can be frought with much frustration and has the potential for complications.

Therefore, there is also a need for a simple, inexpensive, and sterile non-X-ray system that could be used before, and even during, surgery to help detect or localize and sterilely mark a patient's skin. In addition, there is a need to have such system be either disposable or resterilizable for such a surgical procedure. There also is a need to have the system include a sterile marker either attached to or provided with the scanning metal medical device to best locate the device prior to any skin puncture or incision.

In addition, retained foreign bodies following surgery are a source of medical errors and may result in a medical malpractice lawsuit. There is a need for a simple scanning device to detect retained foreign bodies (metal and otherwise). A simple handheld scanner that could detect such retained foreign bodies could be used before closing the surgical wound and especially prior to the patient leaving the operative suite to prevent additional surgeries by identifying the foreign body and allowing the removal of the unintended material(s). Then, the wound/incision could be safely and surgically closed with peace of mind that there is no retained foreign body.

### OBJECTS OF THE INVENTION

It is an object of the invention to provide a handheld sterile system for detecting or identifying hidden objects within the human body prior to surgical exploration, removal, revision, or replacement.

It is also an object of the invention to provide a method for detecting or identifying hidden objects within the human body prior to surgical exploration, removal, revision, or replacement.

It is a further object of the invention to provide a system in which a sterile medical device and marker are used to identify the location of a hidden object prior to or during a surgical procedure.

It is a yet further object of the invention to provide a system in which the sterile medical device comprises a power supply, object detector, monitor/alarm system, and a plastic or rubber waterproof or water resistant casing.

It is a yet further object of the invention to provide a system wherein a handheld medical device can detect metal objects either on or under the skin.

It is a yet further object of the invention to provide a system wherein a handheld medical device can detect ferrous or nonferrous objects.

It is a yet further object of the invention to provide a system of in which the sterile medical device is disposable.

It is a yet further object of the invention to provide a system in which the medical device is sterilizable or resterilizable.

It is a yet further object of the invention to provide a system in which the objects to be identified and/or located and/or detected are made of metal or a material other than metal.

It is a yet further object of the invention to provide a sterile handheld medical system comprising means for scanning and detecting or locating an object hidden in or underneath a patient's skin based on an audible and/or visual feedback system.

It is a yet further object of the invention to provide a sterile handheld medical system also comprising means for marking the location of the object on the skin.

It is a yet further object of the invention to provide a sterile handheld medical system also comprising a tool for removing the object from the patient.

It is a yet further object of the invention to provide a sterile method for scanning and detecting or locating an object hidden in or underneath the skin based on an audible and/or visual feedback system, optionally marking the location of the object on the skin, and then removing the object.

It is a yet further object of the invention to provide a system which comprises a medical scanner or locator, a skin puncturing tool, a digging tool, and an object grabber to securely grab onto the object such that is can be retracted and the object is removed from the human body.

It is a yet further object of the invention to provide a system wherein the digging tool and grabber contain a locating element to hone in on and precisely locate the foreign object.

It is a yet further object of the invention to provide a handheld sterile medical device and system which can scan and detect or localize and mark objects hidden within the human body prior to surgical exploration, removal, revision, or replacement.

It is a yet further object of the invention to provide a device or system wherein objects are detected or located by a method applied at or near the skin surface.

It is a yet further object of the invention to provide a device or system wherein the method comprises ultrasound technology, changes in resistance, resistivity, or the dielectric current, changes in the magnetic field, changes in the electrical current, optical light changes, thermal changes and/or radiant heat, and/or use of a radiofrequency sensor.

It is a yet further object of the invention to provide a device or system of which comprises technology for determining the depth of the object or objects.

It is a yet further object of the invention to provide a device or system which comprises technology for determining the location, size, and depth of a foreign object within the human body.

It is a yet further object of the invention to provide a handheld sterile system for detecting or identifying objects hidden within the human body prior to surgical wound closure, following surgical wound closure, or after surgery to identify and locate a retained foreign body.

It is a yet further object of the invention to provide a sterile method for identifying objects hidden within the human body prior to surgical wound closure, following surgical wound closure, or after surgery to identify and locate a retained foreign body.

It is a yet further object of the invention to provide a system for identifying and locating both foreign objects and blood vessels to increase safety of a surgical procedure by avoiding inadvertent damage to a significant blood vessel that might cause bleeding or other complications.

It is a yet further object of the invention to provide a method for identifying and locating both foreign objects and blood vessels to increase safety of a surgical procedure by avoiding inadvertent damage to a significant blood vessel that might cause bleeding or other complications.

These and other objects of the invention will become more apparent from the description, drawings, and claims below.

### SUMMARY DESCRIPTION OF THE INVENTION

Described herein is a precise method and system for locating any materials, metallic or nonmetallic, hidden on or beneath a patient's skin. More particularly, a system and a method identifies and marks the skin for surgical exploration, identification, removal, revision, and replacement of metal and nonmetal materials located on and/or under the skin. In addition, the scanner could be used to assure that there is no retained foreign body following surgery and prior to wound closure.

Scanners exist for detecting or identifying structures comprised of metal and nonmetal materials that may be hidden beneath a wall. Devices that are typically available in hardware stores, known as stud finders, measure changes in dielectric current and can identify the presence of wood beams or studs beneath a wall. Such stud finders can be tuned and provide feedback to the user to locate wooden beams or studs. In addition, metal scanners can be used to locate both ferrous and nonferrous metals which might be hidden beneath a wall. For example, Zircon makes hardware tools that function as both a stud finder and/or a metal scanner. Their stud finder device can identify the edges of wooden beams beneath a wall, and their metal scanner can identify nails, screws, and pipes that are hidden behind a wall. Nonferrous materials such as copper that are up to two inches deep and ferrous materials such as steel that are up to four inches deep can be identified in this way. The Zircon devices can find (1) studs, (2) metal objects, or (3) both studs and metal objects.

The Zircon system uses a magnetic field and sensor to identify the hidden metal object. A series of light indicators (LEDs) along with an audible sound reflect the proximity of a hidden object. Alternatively, a display monitor can be used to also indicate the proximity of the hidden object.

One example of a device used by carpenters is the Zircon METALLISCANNER® M40 stud finder. This is a handheld carpentry tool, which can detect metal behind a wall (ferrous and nonferrous). It is approximately 8 inches long, handheld, and powered by a 9 volt battery. It uses a series of red lights (LEDs) as well as an audible tone to alert the user that a metal object is located. See, for example, Zircon's U.S. Pat. No. 7,812,722, which describes a hardware store device that can use two modes to detect metal.

The invention herein provides a system and method for sterile scanning and marking on or of the skin the location of metal and nonmetal materials that reside on or under the skin. Such materials may include, for example, an implantable device such as a pacemaker, implantable loop recorder, implantable physiologic monitor, implantable sensor, implantable tracker, implantable communicators, implantable prostheses, implantable GPS systems, pumps, monitors, pacemakers, defibrillator, drug delivery pumps, pins, screws, or other hardware, retained needles or any equipment left from surgery, or an advertent or inadvertent implantable device or material of any sort. In addition, the materials may also include non-implantable materials such as a bullet or shrapnel, which may require surgery for removal or explantation. The system could also be used to scan a patient prior to surgical wound closure to avoid the problem of a retained foreign body and the need for a second surgical procedure to find and remove the retained foreign body. This novel sterile handheld scanner could also be used in the retained foreign body cases to identify the retained foreign material, localize it, and remove it accordingly.

In an embodiment of the invention, a handheld sterile, sterilizable, or resterilizable system is capable of detecting, locating, or identifying hidden objects within the human body prior to surgical exploration, removal, revision, or replacement.

In an embodiment of a method of the invention, a handheld sterile, sterilizable, or resterilizable system is used to detect, locate, or identify hidden objects within the human body prior to surgical exploration, removal, revision, or replacement.

In another embodiment of the invention, a sterile, sterilizable, or resterilizable medical device and marker are used to detect, locate, or identify the location of a hidden object prior to a surgical procedure.

In another embodiment of the invention, the sterile, sterilizable, or resterilizable medical device comprises a power supply, object detector, monitor/alarm system, and a plastic or rubber waterproof or water resistant casing.

In another embodiment of the invention, the handheld medical device can detect metal objects either on or under the skin.

In another embodiment of the invention, the handheld medical device can detect ferrous objects.

In another embodiment of the invention, the handheld medical device can detect nonferrous objects.

In another embodiment of the invention, the sterile, sterilizable, or resterilizable medical device is disposable.

In another embodiment of the invention, the objects to be detected are made of a material other than metal.

In another embodiment of a system of the invention, the system is capable of scanning and detecting an object hidden in or underneath a patient's skin based on an audible and/or visual feedback system.

In another embodiment of a system of the invention, the system is capable of marking the location of said object on the patient's skin.

In another embodiment of a system of the invention, the system comprises a tool capable of removing the object from the patient.

In another embodiment of the invention, a handheld medical system comprises means for scanning and locating an object hidden in or underneath the skin, marking the location of said object on the skin, and finding and then removing the object, based on an audible and/or visual feedback system.

In another embodiment of the invention, a method comprises scanning and detecting or locating an object hidden in or underneath the skin based on an audible and/or visual feedback system, optionally marking the location of said object on the skin, and then removing the object.

In another embodiment of the invention, a system comprises a medical scanner or locator, a skin puncturing tool, a digging tool, and an object grabber to securely grab onto the object such that is can be retracted and the object is removed from the human body.

In another embodiment of the invention, the digging tool and grabber contain the locating element to hone in on and precisely locate the foreign object.

In another embodiment of the invention, a handheld sterile medical device and system can scan and detect or localize and mark objects hidden within the human body prior to surgical exploration, removal, revision, or replacement.

In another embodiment of the invention, objects are located by a method applied at or near the skin surface.

In another embodiment of the invention, an object is detected, located, or identified by ultrasound technology, changes in resistance, resistivity, or the dielectric current, changes in the magnetic field, changes in the electrical current, optical light changes, thermal changes and/or radiant heat, and/or use of a radiofrequency sensor.

In another embodiment of the invention, a device or system comprises technology for determining the depth of the object or objects.

In another embodiment of the invention, a device or system comprises technology for determining the location, size, and depth of a foreign object within the human body.

In another embodiment of the invention, an object or objects within a patient's body are detected, located, or identified prior to surgical wound closure, following surgical wound closure, or after surgery, to identify and locate a retained foreign body.

In another embodiment of the invention, one or more foreign objects or blood vessels are detected, located, or identified to increase safety of a surgical procedure by avoiding inadvertent damage to a significant blood vessel that might cause bleeding or other complications.

In one embodiment of the invention, a scanning device could comprise a rubber or plastic sterile waterproof or water resistant shielded device which encompasses (1) a power supply, (2) a sensor/detector, and (3) monitor and/or alarm (visual and/or audio).

In another embodiment of a medical scanning device of the invention, a sterile marker is included together with the scanning device to mark the skin at the location in which implantable or implanted material is detected.

The scanning methods may include, but are not limited to, methods applied at or near the skin surface, such as ultrasound technology, changes in resistance, resistivity, or the dielectric current, changes in the magnetic field, optical light changes, thermal temperature changes, and/or radiant heat.

In another embodiment of the invention, a light and/or sterile marker is contained within the medical scanning device such that the light would shine at the location where the detection signal is the strongest. Alternatively, the sterile marker could be advanced from a retracted position from the device to mark the skin and then retracted when removed.

In another embodiment of the invention, a surgical tool is attached to a location device such that (1) the skin could be anesthetized and/or (2) the skin could be punctured, and/or (3) the surgical tool could advance underneath the skin towards the indentifiable subcutaneous object, and/or (4) the surgical tool would create a louder signal and/or visual cue helping the location device precisely locate the object, and/or (5) a grabbing tool could grab such object and securely hold it in place, and/or (6) said grabbing object could be retracted into a removal tool and removed out of the human to remove said material(s).

In another embodiment of the invention, the scanner device may have a protective boot or sheath to keep the device sterile and clean. Such a protective boot or sheath may be flexible, rigid, or a combination thereof, preferably from a sterile or sterilizable material such as a polymer.

In another embodiment of the invention, the location of an implanted or subcutaneous object can be marked by, for example, a colored sticker with or without writing or symbols placed on the skin, marking on the skin from a marker comprising indelible ink, or a radiopaque sticker.

In another embodiment of the invention, a method and system for identifying and locating both foreign objects and blood vessels increases the safety of a surgical procedure by avoiding inadvertent damage to a significant blood vessel that might cause bleeding or other complications.

In another embodiment of the invention, the scanning device comprises technology or structure to indicate the depth of an implanted or subcutaneous object. For example, the scanning device may comprise a telescoping rod or member that calibrates the edge of the electromagnetic field, to determine depth with almost pinpoint accuracy. If the telescoping rod is graduated, it could provide an estimate on the depth under the surface of the skin. The shorter the rod, the greater the depth. This will ensure the smallest incision size and allow a doctor to find an object or device in the most expedient manner possible because the doctor will have an estimate of the depth below the epidermis.

The invention herein is directed to a handheld, sterile, sterilizable, or resterilized device. It is within the scope of the invention that the device could be a larger device attached to a console or other structure.

### BRIEF DESCRIPTION OF THE DRAWINGS

FIG. 1 represents a schematic representation of an embodiment of the invention positioned at a distance from an object to be detected. The telescoping rod is retracted;

FIG. 2 represents a schematic representation of the embodiment shown in FIG. 1 where the telescoping rod has been extended; and

FIG. 3 represents a schematic representation of the embodiment shown in FIG. 2 where a sterile barrier has been attached to the telescoping rod.

### DETAILED DESCRIPTION OF THE INVENTION

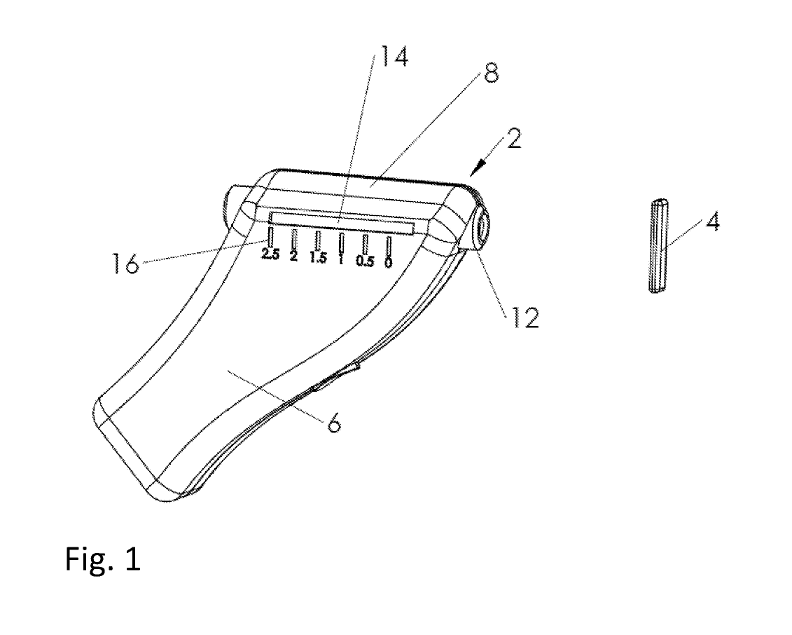

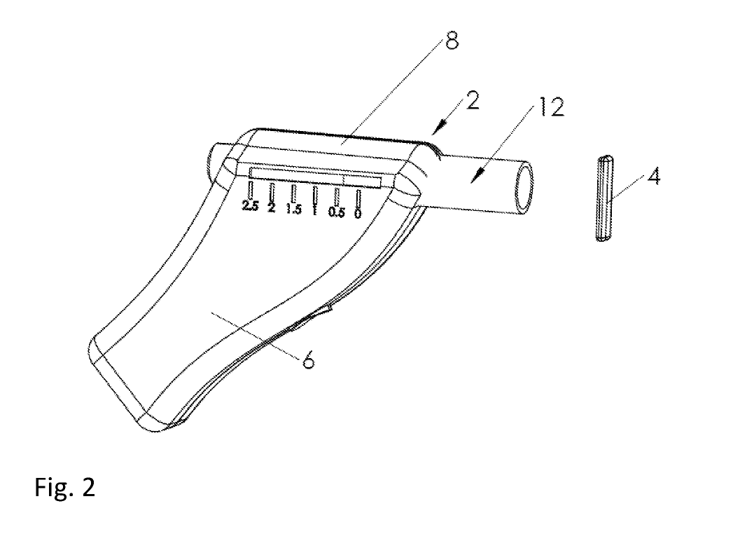

The invention can perhaps be better appreciated by reference to the drawings. In FIG. 1, a device 2 is positioned at a distance from an object 4 to be detected. Device 2 comprises a grippable handle 6 attached to an upper portion 8. Upper portion 8 preferably comprises a telescoping rod 12, shown retracted in FIG. 1. In FIG. 2, telescoping rod 12 is extended.

Upper portion 8 has a lever or other mechanism (not shown) for advancing telescoping rod 12. Upper portion 8 comprises a window 14 with markings 16 that correspond to and measure the extent that telescoping rod 12 is extended.

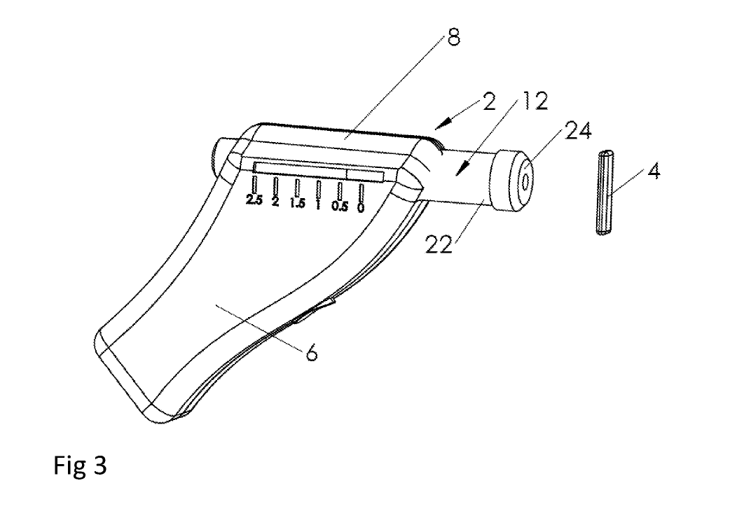

In FIG. 3, telescoping rod 12 has a sterile barrier or tip 20 positioned on its distal end 22. Barrier or tip 20 has a sticker or marker 24 for indicating the location of an object when pressed against a patient's skin (not shown).

Object 4 shown in FIGS. 1 to 3 is a LINQ™ Insertable Cardiac Monitor, available from Medtronic. When a device such as the LINQ device is implanted, its depth is unknown. Telescoping rod 12 can be used to ascertain position and depth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed:

1. A handheld medical system for detecting at least one object within a patient's body based on an audible and/or visual feedback system, prior to surgical exploration, removal, revision, or replacement.

2. The system of claim 1 which is capable of marking the location of the object prior to a surgical procedure.

3. The system of claim 1 which comprises a power supply, object detector, monitor/alarm system, and a plastic or rubber waterproof or water resistant casing.

4. The system of claim 1 which can detect metal or nonmetal objects either on or under the skin.

5. The system of claim 4, wherein the handheld medical device can detect ferrous objects.

6. The system of claim 4, wherein the handheld medical device can detect nonferrous objects.

7. The system of claim 1 which is disposable.

8. The system of claim 1 which sterile, sterilizable, or resterilizable.

9. The system of claim 1 which is comprises a tool for finding and then removing the object.

10. The system of claim 9 which comprises a medical scanner or detector, a skin puncturing tool, a digging tool, and an object grabber to securely grab onto the object so that it can be retracted and removed from the patient's body.

11. The system of claim 10, wherein the digging tool and grabber comprise a locating element to hone in on and precisely locate the foreign object.

12. The system of claim 1, wherein an object is detected by ultrasound technology, changes in resistance, resistivity, or the dielectric current, changes in the magnetic field, changes in the electrical current, optical light changes, thermal changes and/or radiant heat, and/or use of a radiofrequency sensor.

13. The system of claim 1 which comprises technology for determining the depth of the object or objects.

14. The system of claim 1 which comprises technology for determining the location, size, and depth of a foreign object within the patient's body.

15. A system of claim 1 for identifying objects hidden within the human body prior to surgical wound closure, following surgical wound closure, or after surgery to identify and locate a retained foreign body.

16. A system of claim 1 for identifying and locating both foreign objects and blood vessels to increase safety of a surgical procedure by avoiding inadvertent damage to a significant blood vessel that might cause bleeding or other complications.

17. A system of claim 1 for detecting at least one object within a patient's body based on an audible and/or visual feedback system, and for finding and removing the object, prior to surgical exploration, removal, revision, or replacement.

18. A system of claim 1 for detecting at least one object within a patient's body based on an audible and/or visual feedback system, for marking the location of the object, and for finding and removing the object, prior to surgical exploration, removal, revision, or replacement.

19. A method for locating and optionally removing an object within a patient's body prior to surgical exploration, removal, revision, or replacement, which comprises using a medical system of claim 1 to detect the location of the object.

20. The method of claim 19, wherein the location of the object is marked on the patient's skin.

21. The method of claim 19, wherein a tool forming part of the medical system is used to extract the object.

22. The method of claim 19, wherein objects are located by a method applied at or near the skin surface.
